(19) United States
(12) Patent Application Publication
(10) Pub. No.: US 2007/0197607 A1
(43) Pub. Date: Aug. 23, 2007

(54) Continuous Dosing Regimen

(76) Inventors: Gary Gordon, Highland Park, IL (US); Anne E. Hagey, Lake Forest, IL (US); Kysa A. Meek, McHenry, IL (US); Saul H. Rosenberg, Grayslake, IL (US)

Correspondence Address: Robert DeBerardine;D-377/AP6A-1, Abbott Laboratories, 100 Abbott Park Road, Abbott Park, IL, 60064-6008, US

(21) Appl. No.: 11/615,328

(22) Filed: Dec. 22, 2006

Related U.S. Application Data
(63) Continuation-in-part of application No. 10/857,235, filed on May 28, 2004.
Application No. 10/857,235 is a continuation-in-part of application No. 10/842,667, filed on May 10, 2004, now abandoned.
Application No. 10/842,667 is a continuation-in-part of application No. 10/447,588, filed on May 29, 2003, now abandoned.

(51) Int. Cl. A61K 31/44; A61K 31/337

(52) U.S. Cl. 514/352; 514/449

(57) ABSTRACT

The invention relates to pharmaceutical compositions comprising combinations of ABT-751 and anti-cancer drugs. These combinations have additive antitumorigenesis activity. This invention also relates to methods of treatment using the combinations.

This application is a continuation-in-part of U.S. application Ser. No. 10/857,235, filed May 28, 2004, which is a continuation-in-part of U.S. patent application Ser. No. 10/842,667, filed May 10, 2004, which is a continuation-in-part of U.S. application Ser. No. 10/447,588, filed May 29, 2003, the specifications of which are hereby incorporated by reference into this application.

### FIELD OF THE INVENTION

The invention relates to compositions comprising drugs having additive antitumorigenesis activity and methods of treatment using the combinations.

### BACKGROUND OF THE INVENTION

Neoplastic diseases are characterized by the proliferation of cells which are not subject to normal cell growth and are a major cause of death in humans and other mammals. Cancer chemotherapy has provided new and effective drugs for treating these diseases and has also demonstrated that drugs which disrupt the microtubule system of the cytoskeleton are effective in inhibiting the proliferation of neoplastic cells. Accordingly, drugs which disrupt the microtubule system are some of the most effective cancer chemotherapeutic agents in use.

### SUMMARY OF THE INVENTION

One embodiment of this invention, therefore, pertains to a continuous oral dosing schedule for treatment of disease in a human with a therapeutically acceptable amount of a drug, or a therapeutically acceptable salt thereof, which binds to tubulin β-subunits, wherein said dosing schedule lasts for at least five days.

Another embodiment pertains to a continuous oral dosing schedule for treatment of disease in a human with a therapeutically acceptable amount of a drug, or a therapeutically acceptable salt thereof, which binds to tubulin β-subunits, wherein said dosing schedule lasts for at least five days and during which the severity of at least one adverse side effect selected from the group consisting of anemia, alopecia, fluid retention, myelosupression, neuropathy and neutropenia is essentially reduced when compared to the severity of the same side effect coincident with treatment of the substantially same disease with a parenterally administered drug which binds to tubulin β-subunits.

Still another embodiment pertains to a continuous oral dosing schedule for treatment of disease in a human with a therapeutically acceptable amount of a drug which binds to the colchicine site of tubulin β-subunits, or a therapeutically acceptable salt thereof, wherein said dosing schedule lasts for at least five days.

Still another embodiment pertains to a continuous oral dosing schedule for treating disease in a human with a therapeutically acceptable amount of a drug, or a therapeutically acceptable salt thereof, which binds to the colchicine site of tubulin β-subunits, wherein said dosing schedule lasts for at least five days and during which the severity of at least one adverse side effect selected from the group consisting of anemia, alopecia, fluid retention, myelosupression, neuropathy and neutropenia is essentially reduced when compared to the severity of the same side effect coincident with treatment of the substantially same disease with a parenterally administered drug which binds to tubulin β-subunits.

Still another embodiment of this invention, therefore, pertains to a continuous oral dosing schedule for treatment of disease in a human with a therapeutically acceptable amount of N-(2-((4-hydroxyphenyl)amino)pyrid-3-yl)-4-methoxy-benzenesulfonamide, or a therapeutically acceptable salt thereof, wherein said dosing schedule lasts for at least five days.

Still another embodiment pertains to a continuous oral dosing schedule for treatment of disease in a human with a therapeutically acceptable amount of N-(2-((4-hydroxyphenyl)-amino)pyrid-3-yl)-4-methoxybenzenesulfonamide, or a therapeutically acceptable salt thereof, wherein said dosing schedule lasts for at least five days, and during which the severity of at least one adverse side effect selected from the group consisting of anemia, alopecia, fluid retention, myelosupression, neuropathy and neutropenia is essentially reduced when compared to the severity of the same side effect coincident with treatment of the substantially same disease with a parenterally administered drug which binds to tubulin β-subunits.

Still another embodiment pertains to a method for treatment of disease in a human, said method comprising continuously orally administering, for at least five days, a therapeutically acceptable amount of a drug, or a therapeutically acceptable salt thereof, which binds to tubulin β-subunits.

Still another embodiment pertains to a method for treatment of disease in a human, said method comprising continuously orally administering, for at least five days, a therapeutically acceptable amount of a drug, or a therapeutically acceptable salt thereof, which binds to tubulin β-subunits, during which dosing schedule the severity of at least one adverse side effect selected from the group consisting of anemia, alopecia, fluid retention, myelosupression, neuropathy and neutropenia is essentially reduced when compared with treatment of the substantially same disease with a parenterally administered drug which binds to tubulin β-subunits.

Still another embodiment pertains to a method for treatment of disease in a human, said method comprising continuously orally administering, for a time period of at least five days, a therapeutically acceptable amount of a drug, or a therapeutically acceptable salt thereof, which binds to the colchicine site of tubulin β-subunits.

Still another embodiment pertains to a method for treatment of disease in a human, said method comprising continuously orally administering, for at least five days, a therapeutically acceptable amount of a drug, or a therapeutically acceptable salt thereof, which binds to the colchicine site of tubulin β-subunits, during which dosing schedule, the severity of at least one adverse side effect selected from the group consisting of anemia, alopecia, fluid retention, myelosupression, neuropathy and neutropenia is essentially reduced when compared to the severity of the same side effect coincident with treatment of the substantially same disease with a parenterally administered drug which binds to tubulin β-subunits.

Still another embodiment pertains to a method for treatment of disease in a human, said method comprising continuously orally administering, for at least five days, a therapeutically acceptable amount of N-(2-((4-hydroxyphenyl)-amino)pyrid-3-yl)-4-methoxybenzenesulfonamide, or a therapeutically acceptable salt thereof.

Still another embodiment pertains to a method for treatment of disease in a human, said method comprising continuously orally administering, for at least five days, a therapeutically acceptable amount of N-(2-((4-hydroxyphenyl)-amino)pyrid-3-yl)-4-methoxybenzenesulfonamide, or a therapeutically acceptable salt thereof, during which dosing schedule, the severity of at least one adverse side effect selected from the group consisting of anemia, alopecia, fluid retention, myelosupression, neuropathy and neutropenia is essentially reduced when compared to the severity of the same side effect coincident with treatment of the substantially same disease with a parenterally administered drug which binds to tubulin β-subunits.

Still another embodiment pertains to a composition for immediate gastrointestinal release of N-(2-((4-hydroxyphenyl)amino)pyrid-3-yl)-4-methoxybenzenesulfonamide comprising a therapeutically effective amount of N-(2-((4-hydroxyphenyl)amino)pyrid-3-yl)-4-methoxybenzenesulfonamide and an excipient, which composition induces, upon continuous oral ingestion, essentially reduced severity of at least one side effect selected from the group consisting of anemia, alopecia, fluid retention, myelosupression, neuropathy and neutropenia when compared to the severity of the same side effect coincident with treatment of the substantially same disease with a parenterally administered tubulin β-subunit binder.

Still another embodiment pertains to a pharmaceutical composition having therapeutic synergy comprising N-(2-((4-hydroxyphenyl)amino)pyrid-3-yl)-4-methoxybenzenesulfonamide and at lease one cancer drug selected from the group consisting of cisplatin, docetaxel, and 5-fluorouracil.

Still another embodiment pertains to a method of treating cancer in a human comprising administering a therapeutically effective amount of N-(2-((4-hydroxyphenyl)-amino)pyrid-3-yl)-4-methoxybenzenesulfonamide and at least one additional drug selected from the group consisting of cisplatin, docetaxel, and 5-fluorouracil.

Another embodiment pertains to a method of treating cancer in a human comprising administering a therapeutically effective amount of N-(2-((4-hydroxyphenyl)-amino)pyrid-3-yl)-4-methoxybenzenesulfonamide and an antimitotic agent.

Another embodiment pertains to a method of treating cancer in a human comprising administering a therapeutically effective amount of N-(2-((4-hydroxyphenyl)-amino)pyrid-3-yl)-4-methoxybenzenesulfonamide and a taxane.

Another embodiment pertains to a method of treating cancer in a human comprising administering a therapeutically effective amount of N-(2-((4-hydroxyphenyl)-amino)pyrid-3-yl)-4-methoxybenzenesulfonamide and docetaxel.

Another embodiment of this invention pertains to compositions comprising therapeutically effective amounts of an antimitotic agent and N-(2-((4-hydroxyphenyl)amino)pyridin-3-yl)-4-methoxybenzenesulfonamide.

Another embodiment of this invention pertains to compositions comprising therapeutically effective amounts of a taxane and N-(2-((4-hydroxyphenyl)amino)pyridin-3-yl)-4-methoxybenzenesulfonamide.

Another embodiment of this invention pertains to compositions comprising therapeutically effective amounts of docetaxel and N-(2-((4-hydroxyphenyl)amino)pyridin-3-yl)-4-methoxybenzenesulfonamide.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and an antimitotic agent.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and a taxane.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and docetaxel.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and an antimitotic agent.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and a taxane.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and docetaxel.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and an antimitotic agent.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and a taxane.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and docetaxel.

Still another embodiment pertains to methods for treating cancer with at least additive antitumorigenesis in a mammal, said methods comprising administering thereto therapeutically effective amounts of docetaxel and N-(2-((4-hydroxyphenyl)amino)pyridin-3-yl)-4-methoxybenzenesulfonamide.

Another embodiment pertains to a method of treating cancer in a human comprising administering a treatment first with a taxane drug followed by a therapeutically effective amount of N-(2-((4-hydroxyphenyl)-amino)pyrid-3-yl)-4-methoxybenzenesulfonamide.

Another embodiment pertains to a method of treating cancer in a human comprising administering a treatment first with docetaxel followed by a therapeutically effective amount of N-(2-((4-hydroxyphenyl)-amino)pyrid-3-yl)-4-methoxybenzenesulfonamide.

Another embodiment pertains to a method of treating cancer in a human comprising administering a therapeutically effective amount of N-(2-((4-hydroxyphenyl)-amino)pyrid-3-yl)-4-methoxybenzenesulfonamide and a platinum chemotherapeutic.

Another embodiment pertains to a method of treating cancer in a human comprising administering a therapeutically effective amount of N-(2-((4-hydroxyphenyl)-amino)pyrid-3-yl)-4-methoxybenzenesulfonamide and cisplatin.

Another embodiment of this invention pertains to compositions comprising therapeutically effective amounts of a platinum chemotherapeutic and N-(2-((4-hydroxyphenyl)amino)pyridin-3-yl)-4-methoxybenzenesulfonamide.

Another embodiment of this invention pertains to compositions comprising therapeutically effective amounts of cisplatin and N-(2-((4-hydroxyphenyl)amino)pyridin-3-yl)-4-methoxybenzenesulfonamide.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and a platinum chemotherapeutic.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and cisplatin.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and a platinum chemotherapeutic.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and cisplatin.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and a platinum chemotherapeutic.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and cisplatin.

Still another embodiment pertains to methods for treating cancer with at least additive antitumorigenesis in a mammal, said methods comprising administering thereto therapeutically effective amounts of a platinum chemotherapeutic and N-(2-((4-hydroxyphenyl)amino)pyridin-3-yl)-4-methoxybenzenesulfonamide.

Another embodiment pertains to a method of treating cancer in a human comprising administering a treatment first with cisplatin followed by a therapeutically effective amount of N-(2-((4-hydroxyphenyl)-amino)pyrid-3-yl)-4-methoxybenzenesulfonamide.

Another embodiment pertains to a method of treating cancer in a human comprising administering a therapeutically effective amount of N-(2-((4-hydroxyphenyl)-amino)pyrid-3-yl)-4-methoxybenzenesulfonamide and an antimetabolite.

Another embodiment pertains to a method of treating cancer in a human comprising administering a therapeutically effective amount of N-(2-((4-hydroxyphenyl)-amino)pyrid-3-yl)-4-methoxybenzenesulfonamide and 5-fluorouracil(5-FU).

Another embodiment of this invention pertains to compositions comprising therapeutically effective amounts of an antimetabolites and N-(2-((4-hydroxyphenyl)amino)pyridin-3-yl)-4-methoxybenzenesulfonamide.

Another embodiment of this invention pertains to compositions comprising therapeutically effective amounts of 5-FU and N-(2-((4-hydroxyphenyl)amino)pyridin-3-yl)-4-methoxybenzenesulfonamide.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and an antimetabolite.

Another embodiment pertains to a method of treating non-small cell lung cancer in a human comprising administering a therapeutically effective amount of N-(2-((4-hydroxyphenyl)-amino)pyrid-3-yl)-4-methoxybenzenesulfonamide and 5-FU.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and an antimetabolite.

Another embodiment pertains to a method of treating metastatic hormone refractory prostate cancer in a human comprising administering a therapeutically effective amount of N-(2-((4-hydroxyphenyl)-amino)pyrid-3-yl)-4-methoxybenzenesulfonamide and 5-FU.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and an antimetabolite.

Another embodiment pertains to a method of treating breast cancer in a human comprising administering a therapeutically effective amount of N-(2-((4-hydroxyphenyl)-amino)pyrid-3-yl)-4-methoxybenzenesulfonamide and 5-FU.

Another embodiment pertains to a method of treating colon cancer in a human comprising administering a therapeutically effective amount of N-(2-((4-hydroxyphenyl)-amino)pyrid-3-yl)-4-methoxybenzenesulfonamide and 5-FU.

Still another embodiment pertains to methods for treating cancer with at least additive antitumorigenesis in a mammal, said methods comprising administering thereto therapeutically effective amounts of an antimetabolite and N-(2-((4-hydroxyphenyl)amino)pyridin-3-yl)-4-methoxybenzenesulfonamide.

Another embodiment pertains to a method of treating cancer in a human comprising administering a treatment first with 5-FU followed by a therapeutically effective amount of N-(2-((4-hydroxyphenyl)-amino)pyrid-3-yl)-4-methoxybenzenesulfonamide.

### BRIEF DESCRIPTION OF THE FIGURES

FIG. 1 shows the effects of HPM in combination with docetaxel in the MDA-MB 468 flank xenografts grown in female nude mice.

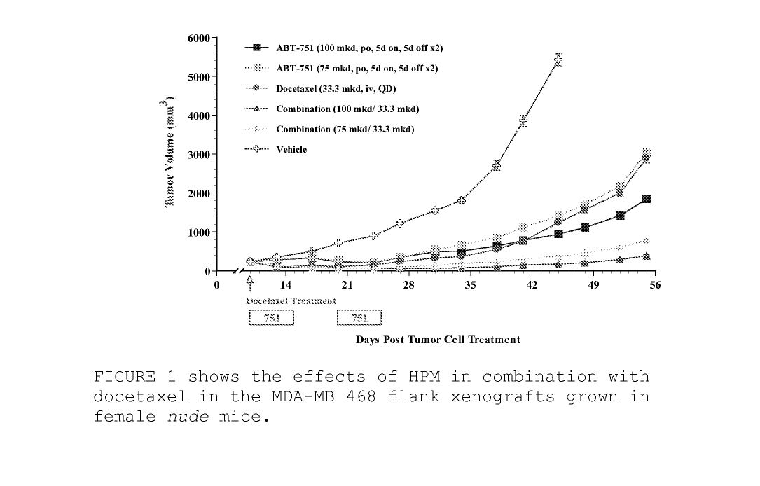

FIG. 2 shows Efficacy of ABT-751 in combination with cisplatin in the Calu-6. flank xenografts grown in nude mice.

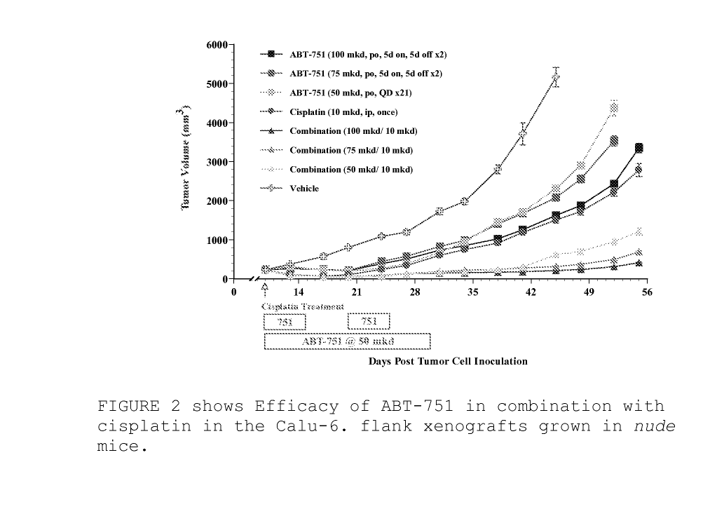

FIG. 3 shows the efficacy of ABT-751 alone and in combination with 5-FU in the HT-29 colon subcutaneous flank xenografts grown in male nude mice.

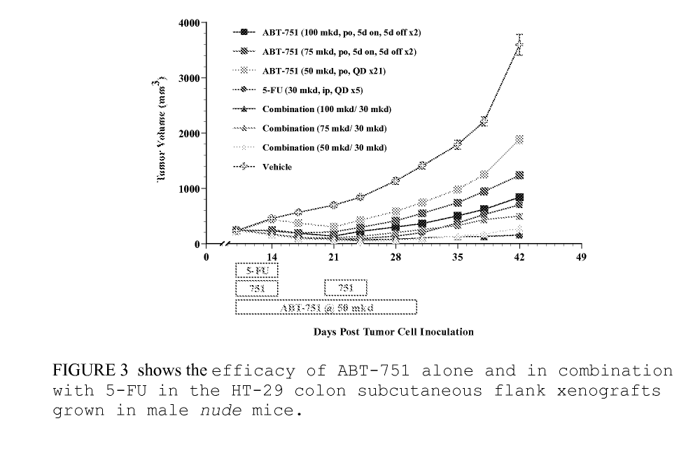

### DETAILED DESCRIPTION OF THE INVENTION

N-(2-((4-Hydroxyphenyl)amino)pyridin-3-yl)-4-methoxybenzenesulfonamide is also referred to herein as HPM, ABT-751 or 751.

The term “additive antitumorigenesis,” as used herein means greater antitumorigenesis than obtained from use of either N-(2-((4-hydroxyphenyl)amino)pyridin-3-yl)-4-methoxybenzenesulfonamide or a co-therapeutic agent.

The term “antimetabolites” includes ALIMTA® (premetrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR, enocitabine, ethnylcytidine, fludarabine, hydroxyurea, 5-fluorouracil (5-FU) alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosate, pelitrexol, pentostatin, raltitrexed, Ribavirin, triapine, trimetrexate, S-1, tiazofurin, tegafur, TS-1, vidarabine, UFT and the like.

The term “antitumorigenesis,” as used herein, means inhibition or reduction of tumor growth.

The term “at least five days,” as used herein, means the time period over which the drug is administered. In a preferred embodiment for the practice of this invention, at least five days means for the first 7 days of a 21 day schedule, for the first 14 days of a 21 day schedule, for he first 15 days of a 21 day schedule, for the first 21 days of a 28 day schedule, for 5 days then cessation for 5 days then continuation for 5 days then cessation for 5 days, i.e. (5 days on/5 days off)×2, and for 7 days then cessation for 7 days then continuation for 7 days then cessation for 7 days, i.e. (7 days on/7 days off)×2.

The term “antimitotic agents” includes batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881, vinflunine, ZK-EPO and the like.

The term “colchicine site binder,” as used herein, means a tubulin β-subunit binder which binds to the colchicine site of the tubulin β-subunits and thereby inhibits the polymerization of tubulin.

A preferred example of a drug which binds to the colchicine site of tubulin β-subunits for the practice of this invention is N-(2-((4-hydroxyphenyl)amino)pyrid-3-yl)-4-methoxybenzenesulfonamide, also referred to herein as HPM. The synthesis of N-(2-((4-hydroxyphenyl)amino)pyrid-3-yl)-4-methoxybenzenesulfonamide is taught in U.S. Pat. No. 5,292,758, column 23, line 61 to column 24, line 12, hereby incorporated by reference into this specification.

The term “cancer,” as used herein, means bone marrow dyscrasias, breast (ductal and lobular) cancer, cervical cancer, colon cancer, leukemia, lung (small cell and non-small cell) cancer, lymphoma, melonoma, mouth and tongue cancer, pancreatic cancer, prostate cancer, rectal cancer, renal cancer, sarcoma, stomach cancer, uterine cancer, and cancers resulting from the metastasis of disease from these areas.

The term “continuous,” as used herein, means at least once per day without missing a day.

The term “disease,” as used herein, means an adverse physiological event. For the practice of this invention, examples of diseases for which drugs which bind to the colchicine site of tubulin β-subunits are useful are gouty arthritis and cancer.

The term “drug,” as used herein, means a compound which is suitable for prevention or treatment of disease or inhibition of one or more adverse physiological events.

Examples of parenterally administered drugs include vinca alkaloids (vincristine, vinblastine, and vinorelbine), taxanes (paclitaxel and docetaxel), 5-fluorouracil, cisplatin, docetaxel, gemcitabine, and colchicine site binders such as colchicine itself which is used to treat gouty arthritis.

The term “essentially reduced,” as used herein in reference to severity of an adverse side effect means at least about 50% of the patient population tested did not experience that side effect at the Grade III or IV level, preferably about 75% of the patient population tested did not experience that side effect at the Grade III or IV level, more preferably about 85% of the patient population tested did not experience that side effect at the Grade III or IV level, even more preferably, about 95% of the patient population tested did not experience that side effect at the Grade III or IV level, and most preferably, 100% of the patient population tested did not experience that side effect at the Grade III or IV level.

The term “platinum chemotherapeutics” includes cisplatin, ELOXATIN® (oxaliplatin) eptaplatin, lobaplatin, nedaplatin, PARAPLATIN® (carboplatin), satraplatin and the like.

The term “taxane,” as used herein, are drugs that inhibits cell growth by stopping cell division. Taxanes are antimitotic agents or mitotic inhibitors. Taxanes include docetaxel and paclitaxel and the like.

The term “therapeutic synergy,” as used herein, means a combination of two or more drugs having a therapeutic effect greater than the additive effect of each respective drug.

The following abbreviations have the defined meanings:

The term “p.o.” means orally.

The term “q.d.” means once per day.

The term “mpk,” as used herein, means milligrams drug per kilogram mammal.

The term “SEM,” as used herein, means standard error of the mean.

The term “T/C,” as used herein, means size of tumor (treated/control).

The term “s.c.,” as used herein, means subcutaneously.

The term “p-value,” as used herein, means confidence level of comparison to control. For example, a p-value less than 0.5 means having greater than 95% confidence that the result did not occur randomly.

Drugs of this invention may be administered, for example, orally, parenterally (intramuscularly, intraperintoneally (i.p), intrasternally, intravenously subcutaneously) or transdermally.

Therapeutically effective amounts of drugs of this invention depend on the recipient of treatment, the cancer being treated and severity thereof, compositions containing them, time of administration, route of administration, duration of treatment, their potency, their rate of clearance and whether or not other drugs are co-administered. The amount of a compound of a drug of this invention used to make a composition to be administered daily to a patient in a single dose or in divided doses is from about 0.05 to about 300 mg/kg (mpk) body weight. Single dose compositions contain these amounts or a combination of submultiples thereof.

Drugs of this invention may be administered with or without an excipient. Excipients include, for example, encapsulating materials or additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising drugs of this invention to be administered parenterally or transdermally include, for example, 1,3-butanediol, castor oil, corn oil, cottonseed oil, dextrose, 5% glucose in water (D5W), germ oil, groundnut oil, isotonic sodium chloride solution (0.9% sodium chloride in water), liposomes, oleic acid, olive oil, peanut oil, Ringer's solution, safflower oil, sesame oil, soybean oil, U.S.P. or, water, mixtures thereof and the like.

Binding affinities of N-(2-((4-hydroxyphenyl)amino)pyrid-3-yl)-4-methoxybenzenesulfonamide, vinblastine, and paclitaxel were evaluated using the competition of [³H]colchicine to biotinylated bovine brain tubulin in a scintillation proximity assay.

TABLE 1
Inhibition Constants of ABT-751 and Other Tubulin
β-Subunit Binders in Binding Experiments with Bovine
Brain Tubulin

| Compound | Kᵢ (μM) |
|---|---|
| ABT-751 | 2.60 (n = 4) |
| colchicine | 0.78 (n = 7) |
| paclitaxel | >100 (n = 4) |
| vinblastine | >100 (n = 4) |

The data in TABLE 1 demonstrate that N-(2-((4-hydroxyphenyl)amino)pyrid-3-yl)-4-methoxybenzenesulfonamide displaces [³H]colchicine from the colchicine site of tubulin β-subunits and is therefore a colchicine site binder.

The data in TABLE 1 also demonstrate that vinblastine and paclitaxel do not displace [³H]colchicine, are therefore not colchicine-site binders, and therefore must bind to tubulin β-subunits sites which are different from the colchicine binding site.

N-(2-((4-hydroxyphenyl)amino)pyrid-3-yl)-4-methoxybenzenesulfonamide is a colchicine site binder and exemplifies drugs which are useful for treatment of diseases which may be treated with colchicine-site binders other than colchicine itself.

The effectiveness of colchicine-site binders as drugs which are useful for treatment of diseases in humans depends on variables such as the composition comprising the drug, its route of administration, the amount of drug administered, and the dosing schedule. This invention pertains to an unexpected and surprising combination of variables which lead to a favorable therapeutic event with a sufficient reduction in the severity of at least one adverse side effect selected from the group consisting of anemia, alopecia, fluid retention, myelosupression, neuropathy and neutropenia as compared to the same side effect coincident with treatment of the substantially same disease with a parenterally administered drug which binds to tubulin β-subunits.

M5076 is a transplantable murine reticulum cell sarcoma. N-(2-((4-hydroxyphenyl)amino)pyrid-3-yl)-4-methoxybenzenesulfonamide exhibited significant antitumor activity in this syngeneic flank tumor model when administered orally once a day for 5 days. At its approximate MTD of 150 mg/kg for 5 days, N-(2-((4-hydroxyphenyl)amino)pyrid-3-yl)-4-methoxybenzenesulfonamide significantly inhibited tumor growth with T/C (tumor mass of test group divided by tumor mass of control group) and ILS (percent increase in life span) values of 13 and 42%, respectively. In contrast, this model was resistant to paclitaxel. Vincristine and doxorubicin were only marginally active (ILS=17% and 13% respectively), while this model proved sensitive to cyclophosphamide.

N-(2-((4-hydroxyphenyl)amino)pyrid-3-yl)-4-methoxybenzenesulfonamide was evaluated against C26 colon tumors grown in the flank of CDF-1 mice. While only marginally active when administered on a 5-day schedule, extended dosing produced a significant antitumor response that was equivalent to that achieved with BCNU at the MTD. Paclitaxel was not efficacious against this tumor.

Apc^{Min} (Min) mice are models for genetically inherited intestinal cancer. These mice carry a dominant germline mutation in the Apc tumor suppressor gene that predisposes them to the development of numerous (>50) tumors throughout the intestinal tract.

N-(2-((4-hydroxyphenyl)amino)pyrid-3-yl)-4-methoxybenzenesulfonamide given orally on a once-a-day schedule for 28 days at 150 mg/kg/day led to a significant reduction in tumor burden in Min mice. The average tumor number for treated mice was 49.8 compared to 73.3 for vehicle controls. Drug treatment was initiated at an age when the tumors were well-established. These results indicate that N-(2-((4-hydroxyphenyl)amino)pyrid-3-yl)-4-methoxybenzenesulfonamide shows significant in vivo activity in a spontaneous model of intestinal tumorigenesis.

(2-((4-hydroxyphenyl)amino)pyrid-3-yl)-4-methoxybenzenesulfonamide as a single agent demonstrated antitumor activity in multiple human tumor xenograft models in vivo. In several in vivo xenograft models (flank and orthotopic), once-a-day dosing of (2-((4-hydroxyphenyl)amino)pyrid-3-yl)-4-methoxybenzenesulfonamide demonstrated equal or greater efficacy compared to twice-a-day dosing. This superior QD efficacy was confirmed in a murine syngeneic model. Thus, administration of (2-((4-hydroxyphenyl)amino)pyrid-3-yl)-4-methoxybenzenesulfonamide once a day appears to be sufficient to achieve maximal efficacy. In combination with 5-FU, cisplatin, docetaxel, and gemcitabine, (2-((4-hydroxyphenyl)amino)pyrid-3-yl)-4-methoxybenzenesulfonamide demonstrated an increase in antitumor activity in the HT-29 colon, Calu-6 NSCLC, MDA-MB-468 breast, and MiaPaCa2 pancreatic xenograft models respectively, compared to single agent alone.

NCI-H460 is a human non-small cell lung carcinoma derived cell line. It is MDR negative, has wild type p53, and contains an oncogenic K-ras mutation. (2-((4-hydroxyphenyl)amino)pyrid-3-yl)-4-methoxybenzenesulfonamide showed good efficacy in the NCI-H460 xenograft model. The mean tumor volume at day 13 was significantly different than the vehicle control (T/C=58%). (2-((4-hydroxyphenyl)amino)pyrid-3-yl)-4-methoxybenzenesulfonamide also caused a delay in tumor growth with an ILS value of 32%. Paclitaxel and vincristine both lacked activity in this assay.

HCT-15 is a human colon carcinoma derived cell line. It is MDR positive, and expresses both mutant p53 and oncogenic K-ras. The HCT-15 cell line has one of the highest levels of mdr-1/P-glycoprotein expression of cells from the NCI tumor cell line panel. Paclitaxel and vincristine, which are both substrates for P-glycoprotein drug efflux pump, were not efficacious, while (2-((4-hydroxyphenyl)amino)pyrid-3-yl)-4-methoxybenzenesulfonamide was effective in inhibiting tumor growth.

TABLE 2
Effect of Cytotoxic Agents on the Growth of HCT-15
Human Colon Carcinoma Xenografts

| | | | T/C^{b} | |
|---|---|---|---|---|
| | Dose^{a} | Route | (no. | |
| Compound | (mg/kg/d) | Schedule | trials) | % ILS^{c} |
| ABT-751 | 50 | PO, QD, | 50 (2) | 34 |
| | | days 1-8 | | |

(2-((4-hydroxyphenyl)amino)pyrid-3-yl)-4-methoxybenzenesulfonamide inhibited the growth of a variety of human tumor xenografts that were allowed to grow into established tumors prior to the initiation of treatment. As summarized below, activity was seen against established tumors derived from colon, breast and lung carcinomas. (2-((4-hydroxyphenyl)amino)pyrid-3-yl)-4-methoxybenzenesulfonamide was also active against a human pancreatic tumor xenograft grown in the orthotopic site.

TABLE 3
In vivo Activity of ABT-751 Against Staged and
Orthotopic Human Tumor Xenografts

| | Oral | | | |
|---|---|---|---|---|
| | Dose | | | |
| Cell line | (mg/kg/d) | Schedule | % T/C | % ILS |
| Tumor Models | | | | |
| HT-29 colon | 100 | QD, d 10-14, | 28 | 75 |
| carcinoma | | 20-24 | | |
| | 75 | QD, d 10-14, | 43 | 50 |
| | | 20-24 | | |
| | 50 | QD, d 1-21 | 55 | 36 |
| Calu-6 lung | 100 | QD, d 10-14, | 37 | 65 |
| carcinoma | | 20-24 | | |
| | 75 | QD., d 10-14, | 50 | 58 |
| | | 20-24 | | |
| | 50 | QD, d 1-21 | 54 | 58 |
| MDA-MB-468 | 100 | QD, d 10-14, | 25 | 78 |
| breast | | 20-24 | | |
| carcinoma | | | | |
| | 75 | QD, d 10-14, | 33 | 52 |
| | | 20-24 | | |
| | 50 | QD, d 1-21 | 46 | 41 |
| Orthotopic | | | | |
| Tumor Model | | | | |
| MiaPaCa-2 | 100 | QD, d 1-5, | 56 | n.d. |
| pancreatic | | 11-15 | | |
| | 75 | QD, d 1-5, | 79 | n.d. |
| | | 11-15 | | |
| HT-1376 bladder | 100 | QD, d 1-5, | 63 | n.d. |
| | | 11-15 | | |
| | 100 | b.i.d., d 1-5, | 86^{e} | n.d. |
| | | 11-15 | | |

(2-((4-hydroxyphenyl)amino)pyrid-3-yl)-4-methoxybenzenesulfonamide also showed antitumor activity against a variety of other human tumor xenografts in nude mice.

Antitumor activity was also seen in tumors derived from gastric, lung, breast, and oral carcinomas.

TABLE 4
Antitumor Activity of ABT-751 Against Human Tumor
Xenografts

| | | | |
|---|---|---|---|
| | | Dose | |
| Tumor Lines | Schedule | (mg/kg/day) | T/C (%) |

Gastric Cancer

| | | | |
|---|---|---|---|
| H-81 | Q5D × 5 | 300 | 40 |
| H-111 | Q1D × 19 | 80 | 36 |
| H-154 | Q5D × 5 | 300 | 71 |
| SC-2 | Q1D × 19 | 120 | 28 |
| SC-6 | Q5D × 5 | 500 | 22 |

Colorectal Cancer

| | | | |
|---|---|---|---|
| H-143 | Q5D × 5 | 300 | 17 |
| COLO320DM | Q1D × 19 | 120 | 42 |
| WiDr | Q1D × 20 | 100 | 22 |

Lung Cancer

| | | | |
|---|---|---|---|
| LC-376 | Q5D × 5 | 300 | 18 |
| LC-6 | Q5D × 5 | 450 | 31 |
| LC-11 | Q1D × 8 | 150 | 93 |
| LX-1 | Q1D × 20 | 120 | 37 |

Breast Cancer

| | | | |
|---|---|---|---|
| H-31 | Q1D × 19 | 80 | 13 |
| MX-1 | Q5D × 5 | 450 | 21 |

Oral Cancer

| | | | |
|---|---|---|---|
| KB | Q1D × 20 | 100 | 15 |

(2-((4-hydroxyphenyl)amino)pyrid-3-yl)-4-methoxybenzenesulfonamide was evaluated in 57 cancer subjects in single dose (16 subjects) and 5-day repeated dose regimens (41 subjects). The doses administered in the single dose segment were 80 to 480 mg/m²/day. In the 5-day repeated dose regimen (2-((4-hydroxyphenyl)amino)pyrid-3-yl)-4-methoxybenzenesulfonamide was given at 30 to 240 mg/m²/day for a single cycle. Pharmacokinetic data indicated that (2-((4-hydroxyphenyl)amino)pyrid-3-yl)-4-methoxybenzenesulfonamide plasma concentrations increased rapidly after dosing. The drug was eliminated with a half-life between 4 and 16 hours. The AUC increased proportionally with dose over the range of 30 to 480 mg/m²/day with no apparent accumulation. Adverse drug reactions from the single dose and 5-day repeated dose segments included nausea and vomiting, diarrhea, epigastric pain, ileus and evidence of peripheral neuropathy. For the single dose segment, the dose limiting toxicity (DLT) was Grade 3 peripheral neuropathy in 1 of 5 subjects at 480 mg/m²/day. In the 5-day repeated dose regimen the dose limiting toxicities were Grade 3 peripheral neuropathy in 1 of 4 subjects at 210 mg/m²/day and Grade 4 intestinal paralysis in 1 of 4 subjects at 210 mg/m²/day and in 1 of 6 subjects at 240 mg/m²/day.

In the 7-day QD regimen, the 250 mg QD dose has been determined to be the MTD, as dose limiting toxicities of peripheral neuropathy/ileus were reported in 2 of 6 subjects at the 300 mg QD dose. The MTD of the QD regimen given for 21 days was determined to be 200 mg as dose limiting toxicities of fatigue, anorexia and suspect small bowel obstruction were observed in 2/3 subjects in the 250 mg dose group.

A review of the safety data demonstrates no significant myelosuppression, renal or hepatic toxicity reported in any of the three studies.

In accordance with compositions, the tubulin β-subunit binders of this invention can be administered with or without an excipient. Excipients include encapsulating materials or additives such as absorption accelerators, antioxidants, binders, buffers, coating agents, coloring agents, diluents, disintegrants, emulsifiers, extenders, fillers, flavoring agents, humectants, lubricants, perfumes, preservatives, propellants, releasing agents, sterilizing agents, sweeteners, solubilizers, wetting agents and mixtures thereof. Excipients for solid dosage forms the tubulin β-subunit binders of this invention to be administered orally include agar, alginic acid, aluminum hydroxide, benzyl alcohol, benzyl benzoate, 1,3-butylene glycol, castor oil, cellulose, cellulose acetate, cocoa butter, corn starch, corn oil, cottonseed oil, ethanol, ethyl cellulose, ethyl laureate, ethyl oleate, gelatin, germ oil, glucose, glycerol, groundnut oil, isopropanol, isotonic saline, lactose, magnesium hydroxide, magnesium stearate, malt, olive oil, peanut oil, potassium phosphate salts, potato starch, propylene glycol, Ringer's solution, talc, tragacanth, water, safflower oil, sesame oil, sodium carboxymethyl cellulose, sodium lauryl sulfate, sodium phosphate salts, soybean oil, sucrose, tetrahydrofurfuryl alcohol and mixtures thereof. Excipients for the tubulin β-subunit binders of this invention to be administered ophthalmically or orally in liquid dosage forms include 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water and mixtures thereof. Excipients for thetubulin β-subunit binders of this invention to be administered osmotically include chlorofluorohydrocarbons, ethanol, water and mixtures thereof. Excipients for the tubulin β-subunit binders of this invention to be administered parenterally include 1,3-butanediol, castor oil, corn oil, cottonseed oil, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof. Excipients for the tubulin β-subunit binders of this invention to be administered rectally or vaginally include cocoa butter, polyethylene glycol, wax and mixtures thereof.

A preferable excipient for the practice of this invention using N-(2-((4-hydroxyphenyl)amino)pyrid-3-yl)-4-methoxybenzenesulfonamide is shown hereinbelow.

TABLE 5
Formulation of N-(2-((4-hydroxyphenyl)amino)pyrid-3-yl)-4-
methoxybenzenesulfonamide

| Ingredient | % w/w | Purpose |
|---|---|---|
| ABT-751 | 30.0 | |
| microcrystalline | 15.8 | Filler |
| cellulose | | |
| NF (Avicel ® PH101) | | |
| lactose monohydrate | 28.0 | Filler |
| povidone (USP, | 8.0 | Binder |
| K29-32) | | |
| croscarmellose Na | 18.0 | Disintegrant |
| water | sufficient quantity | Binder Liquid |
| magnesium stearate | 0.2 | Lubricant |

A mixture of microcrystalline cellulose, N-(2-((4-hydroxyphenyl) amino) pyrid-3-yl)-4-methoxybenzenesulfonamide, lactose, and croscarmellose were granulated with a solution of povidone in water, dried, and milled. The milled product was blended with magnesium stearate.

The doses herein were made by filling capsules with the appropriate amount of blended product.

In accordance with routes of administration, the tubulin β-subunit binders of this invention may be administered orally, ophthalmically, osmotically, parenterally (subcutaneously, intramuscularly, intrasternally, intravenously), rectally, topically, transdermally, or vaginally. Orally administered solid dosage forms can be administered as capsules, dragees, granules, pills, powders, or tablets. Ophthalmically and orally administered dosage forms may be administered as elixirs, emulsions, microemulsions, suspensions, or syrups. Osmotically and topically administered dosage forms may be administered as creams, gels, inhalants, lotions, ointments, pastes, or powders. Parenterally administered dosage forms may be administered as aqueous or oleaginous suspensions. Rectally and vaginally dosage forms may be administered as creams, gels, lotions, ointments, or pastes.

For the practice of this invention, it is meant to be understood that while administration of drugs which bind to other than the colchicine binding site of tubulin β-subunits are preferentially administered parenterally, oral administration of drugs which bind to the colchicine binding site of tubulin β-subunits is more preferable than parenteral administration of the same drug.

The therapeutically acceptable amounts of the tubulin β-subunit binders of this invention and their dosing schedules depend on the recipient of treatment, the disease being treated and the severity thereof, the composition containing the tubulin β-subunit binder, the time of administration, the route of administration, the potency of the tubulin β-subunit binder, the rate of clearance of the tubulin β-subunit binder, and whether or not another drug is co-administered.

The daily amount of N-(2-((4-hydroxyphenyl)amino)pyrid-3-yl)-4-methoxybenzenesulfonamide to be administered orally in a continuous, once daily dose to adult patients having refractory solid tumors, is about 50 mg, about 100 mg, about 150 mg, about 200 mg, about 250 mg, or about 300 mg.

Preferably, about 250 mg of N-(2-((4-hydroxyphenyl)amino)pyrid-3-yl)-4-methoxybenzenesulfonamide is continuously administered orally once per day (QD) to adult patients having refractory solid tumors for the first 7 days of a 21 day schedule.

Preferably, about 200 mg of N-(2-((4-hydroxyphenyl)amino)pyrid-3-yl)-4-methoxybenzenesulfonamide is continuously administered orally once per day to adult patients having refractory solid tumors for the first 21 days of a 28 day schedule.

Preferably, about 200 mg of N-(2-((4-hydroxyphenyl)amino)pyrid-3-yl)-4-methoxybenzenesulfonamide is administered continuously orally once per day to adult patients having breast lung, kidney, or colon cancer, is for the first 21 days of a 28 day dosing schedule.

The daily amount of N-(2-((4-hydroxyphenyl)amino)pyrid-3-yl)-4-methoxybenzenesulfonamide to be administered orally in a continuous once per day dose to pediatric patients having refractory solid tumors, may be about 100 mg/mm², about 130 mg/mm², about 165 mg/mm², about 200 mg/mm², or about 250 mg/mm².

The daily amount of N-(2-((4-hydroxyphenyl)amino)pyrid-3-yl)-4-methoxybenzenesulfonamide to be administered orally in a continuous once per day dose to adult patients having refractory hematologic malignancies, may be about 100 mg/mm², about 125 mg/mm², and about 150 mg/mm².

The daily amount of N-(2-((4-hydroxyphenyl)amino)pyrid-3-yl)-4-methoxybenzenesulfonamide to be administered orally in continuous, twice daily (BID) doses to adult patients having refractory solid tumors, may be about 25 mg, about 50 mg, about 75 mg, about 100 mg, about 125 mg, about 150 mg, about 175 mg, about 200 mg, about 250 mg, or about 300 mg.

Preferably, about 175 mg of N-(2-((4-hydroxyphenyl)amino)pyrid-3-yl)-4-methoxybenzenesulfonamide is administered orally twice per day to adult patients having refractory solid tumors for the first 7 days of a 21 day schedule.

The daily amount of N-(2-((4-hydroxyphenyl)amino)pyrid-3-yl)-4-methoxybenzenesulfonamide to be administered orally in continuous twice per day doses to adult patients having refractory hematologic malignancies may be about 75 mg/mm², about 100 mg/mm², 125 mg/mm², 150 mg/mm², and 175 mg/mm².

The daily amount of N-(2-((4-hydroxyphenyl)amino)pyrid-3-yl)-4-methoxybenzenesulfonamide to be administered in continuous twice per day doses to pediatric patients having refractory solid tumors may be about 100 mg/mm² and about 130 mg/mm².

N-(2-((4-hydroxyphenyl)amino)pyrid-3-yl)-4-methoxybenzenesulfonamide may also be useful in the treatment of disease when used alone or in combination with other therapies. For example, when used for the treatment of cancer, the compounds of the invention may be administered alone or in combination with radiotherapy, hormonal agents, antibodies, antiangiogenics, COX-2 inhibitors, or other chemotherapeutic agents (cytotoxic or cytostatic) such as cisplatin, 5-fluorouracil, taxotere, docetaxel and gemcitabine.

Accordingly, N-(2-((4-hydroxyphenyl)amino)pyrid-3-yl)-4-methoxybenzenesulfonamide in combination with cisplatin (Calu-6 NSCLC), docetaxel (MDA-MB-468) or 5-FU (HT-29) showed equal to or greater than additive efficacy compared to single agents alone.

To evaluate the pharmacokinetics of representative extended dosing schedules of N-(2-((4-hydroxyphenyl)amino)pyrid-3-yl)-4-methoxybenzenesulfonamide, 43 patients were enrolled. The tumor types studied were colorectal (23), sarcoma (5), mesothelioma (3), salivary gland (2), endometrial (2), unknown (2), hepatoma (1), melanoma (1), renal cell (1), lung (1), ovary (1), and granulosa cell (1). Patients were treated once or twice per day for 21 days with N-(2-((4-hydroxyphenyl)amino)pyrid-3-yl)-4-methoxybenzenesulfonamide followed by a 7-day period where no drug was received. Doses were escalated by 50 mg/day (25 mg twice per day). Three patients were initially treated at each dose level. If dose-limiting toxicity was observed in cycle one, three more patients were added to that dosing schedule. If additional patients experienced dose-limiting toxicity, on occasion the dose level was expanded to nine patients to further assess tolerability. Response assessment was performed every two cycles.

For 134 patients tested, at doses of about 200 mg QD, 250 mg QD, 300 mg QD, 125 mg BID, 150 mg BID, and 175 mg BID, 16 reported anemia, of which 11 were Grades I or II and 5 were Grades (III or IV); 1 reported Grade I or II alpoecia; 8 reported Grade I or II neutropenia; and none reported fluid retention, for which Grade I is defined as mild, Grade II is defined as moderate, Grade III is defined as severe, Grade IV is defined as life threatening.

The foregoing is merely illustrative of the invention and is not intended to limit the invention to the disclosed embodiments. Variations and changes obvious to one skilled in the art are intended to be within the scope and nature of the invention which are defined in the appended claims.

The following examples are presented to provide what is believed to be the most useful and readily understood description of procedures and conceptual aspects of this invention.

In the following studies “HPM” is ABT-751 which is N-(2-((4-hydroxyphenyl) amino)pyrid-3-yl)-4-methoxybenzenesulfonamide.

### EXAMPLE 1

### Preclinical Non-Small Cell Lung Cancer Study: HPM and Docetaxel

Male scid mice (Charles River Labs.) were injected subcutaneously with 0.2 mL of 2×10⁶ Calu-6 cells (1:1 with matrigel) on study day 0. The cellular implantation site was the right flank, and all mice were ear tagged. On day 13, tumors were size matched to 247 mm³ and animals were placed into the therapy groups outlined in the study design below. All treatments were initiated on day 13 post tumor inoculation. The tumors were measured with calipers 2 times per week after tumors were palpable. Tumor volumes were calculated according to the formula V=L×W²/2 (V: volume, mm³; L: length, mm. W: width, mm). Mouse group weights were taken 2 times per week to monitor weight loss from toxicity or excessive tumor burden. The mice were humanely euthanized when the tumor volumes reached a predetermined size. The study design was as follows:
1. HPM vehicle and docetaxel at 20 mg/kg/day.
2. HPM vehicle and docetaxel at 10 mg/kg/day.
3. HPM at 100 mg/kg/day and docetaxel vehicle.
4. HPM at 75 mg/kg/day and docetaxel vehicle.
5. HPM at 100 mg/kg/day and docetaxel at 20 mg/kg/day.
6. HPM at 100 mg/kg/day and docetaxel at 10 mg/kg/day.
7. HPM at 75 mg/kg/day and docetaxel at 20 mg/kg/day.
8. HPM at 75 mg/kg/day and docetaxel at 10 mg/kg/day.
9. HPM vehicle and docetaxel vehicle.

HPM was dosed orally on a (q.d.×5, 5 days off)×2 schedule. The drug was formulated in 1% HCl, 4% ethanol, and 95% D5W. Docetaxel was dosed intravenously, (q.10d.)×2 and formulated in saline.

The MTD of docetaxel on a q.10d.×2 schedule was 25 mg/kg/day. In this trial a q.10d. at 20 mg/kg/day had only 8% maximum wt loss, which provided an acceptable window for drug combinations. The MTD of HPM is 100 mg/kg/day using the schedule shown hereinabove. In combination with docetaxel, HPM demonstrated greater than additive responses. The results are shown in TABLE 6.

TABLE 6
In vivo efficacy of HPM with docetaxel in the Calu-6
flank xenograft model. HPM was dosed p.o. 5 days on, 5 days
off for 2 cycles while docetaxel was administered i.v. on days
1 and 11.

| | Dose | | | |
|---|---|---|---|---|
| Compound | (mg/kg/day) | % TGD^{a} | % TGD^{b} | % TGD^{c} |
| HPM/docetaxel | 0/20 | 37*** | — | 18*** |
| HPM/docetaxel | 0/10 | 16 | −15 | — |
| HPM/docetaxel | 100/0 | 16 | −15 | 0 |
| HPM/docetaxel | 75/0 | 16 | −15 | 0 |
| HPM/docetaxel | 100/20 | 133*** | 70*** | 100*** |
| HPM/docetaxel | 100/10 | 54*** | 12 | 32*** |
| HPM/docetaxel | 75/20 | 137*** | 73*** | 104*** |
| HPM/docetaxel | 75/10 | 51*** | 10 | 29** |
| Combo vehicles | 0/0 | | | |

^{a}Median % TGD (tumor growth delay) increase compared to vehicle in time to 1.5 cc tumor; p values calculated from Kaplan Meier Logrank analysis
^{b}Median % TGD increase compared to docetaxel 20 mkd in time to 1.5 cc tumor; p values calculated from Kaplan Meier Logrank analysis
^{c}Median % TGD increase compared to docetaxel 10 mkd in time to 1.5 cc tumor; p values calculated from Kaplan Meier Logrank analysis
^{d}p values vs. vehicle,
**< 0.01,
***< 0.001

### EXAMPLE 2

### Preclinical Prostate Cancer Study: HPM and Docetaxel

Male scid mice (Charles Rivers Labs) were injected subcutaneously with 0.2 mL of 2×10⁶ PC-3 cells (1:1 matrigel) on study day 0. The cellular implantation site was the right flank, and all mice were ear tagged. Tumors were size matched to 209 mm³, and animals were placed into the therapy groups shown in the study design hereinbelow. Both HPM and docetaxel treatments were initiated on day 15. The tumors were measured with calipers 2 times per week after tumors were palpable. Tumor volumes were calculated according to the formula V=L×W²/2 (V: volume, mm³; L: length, mm; W: width, mm). The mice were humanely euthanized when the tumor volumes reached a predetermined size. The study design was as follows:
1. HPM at 100 mg/kg/day p.o., q.d. on a 5 days on 5 days off schedule for 2 cycles and docetaxel vehicle (1% ethanol in D5W)
2. HPM Vehicle-1 eq. 1N HCl, 4% ethanol, 95% D5W and docetaxel—33 mg/kg/day, i.v., q.d×1
3. HPM Vehicle-1 eq. 1N HCl, 4% ethanol, 95% D5W and docetaxel—16.5 mg/kg/day, i.v., q.d×1
4. HPM at 100 mg/kg/day was administered in combination with docetaxel at 33 mg/kg/day single dose
5. HPM at 100 mg/kg/day was administered in combination with docetaxel at 16.5 mg/kg/day single dose
6. Vehicle for HPM and vehicle for docetaxel.

In combination with docetaxel, HPM exhibited additive antitumor effects. The results are shown in TABLE 3A. Although some toxicity was noted in the combination groups (Table 3B) much of the toxicity was noted long after the dosing periods, which may be attributed to other factors outside of drug toxicities such as tumor burden.

TABLE 7
In vivo efficacy of HPM in combination with docetaxel in
the PC-3 prostate flank xenografts grown in male scid mice.

| | Dose | Route | | |
|---|---|---|---|---|
| Compound | (mg/kg/day) | Schedule | % TGD^{a} | % TGD^{b} |
| HPM/ | 100/0 | p.o., q.d.(5 days on, | 77**^{c} | −6 |
| docetaxel | | 5 days off) × 2/ | | |
| | | i.v., q.d. × 1 | | |
| HPM/ | 100/16.5 | p.o., q.d.(5 days on, | 143** | 30* |
| docetaxel | | 5 days off) × 2/ | | |
| | | i.v., q.d. × 1 | | |

^{a}Mean % TGD increase compared to vehicle in time to 0.7 cc
^{b}Mean % TGD increase compared to docetaxel alone in time to 0.7 cc
^{c}p values vs. vehicle,
*< 0.01,
**< 0.001

### EXAMPLE 3

### Preclinical Breast Cancer Study: HPM and Docetaxel

Tumor cells derived from serially passaged tumor fragments were inoculated s.c. in female nude mice on day 0. On day 10, mice bearing established tumors were size matched at about 231 mm³ and divided into the following groups:
1. HPM at 100 mg/kg/day (p.o., q.d., 5 days on, 5 days off ×2)
2. HPM at 75 mg/kg/day (p.o., q.d., 5 days on, 5 days off ×2)
3. HPM at 0 mg/kg/day (p.o., q.d., 5 days on, 5 days off ×2)
4. Docetaxel at 33.3 mg/kg/day (i.v., q.d. ×1)
5. Docetaxel at 0 mg/kg/day (i.v., q.d. ×1)
6. Combination therapy: HPM at 100 mg/kg/day and docetaxel at 33.3 mg/kg/day
7. Combination therapy: HPM at 75 mg/kg/day and docetaxel at 33.3 mg/kg/day
8. Combination Vehicles

As a single agent, HPM at 100 and 75 mg/kg/day 5 days on, 5 days off for 2 cycles demonstrated dose-dependent antitumor activity in the MDA-MB 468 xenograft model. In combination with docetaxel at 33.3 mg/kg/day, HPM at 100 mg/kg/day demonstrated at least additive responses with both doses of HPM tested. The results are shown in TABLE 8.

TABLE 8
In vivo efficacy of HPM alone and in combination with
docetaxel in the MDA-MB-468 breast subcutaneous flank
xenografts grown in female nude mice.

| | Dose | Route | |
|---|---|---|---|
| Compound | (mg/kg/day) | Schedule | % TGD^{a} |
| HPM | 100 | p.o., q.d., (5 days | 78***^{b} |
| | | on, 5 days off) × 2 | |
| | 75 | | 52*** |
| Docetaxel | 33.3 | i.v., q.d × 1 | 67*** |
| HPM/docetaxel | 100/33.3 | p.o., q.d., (5 days | 170***^{c,d} |
| | | on, 5 | |
| | 75/33.3 | days off) × 2/i.p., | 130***^{c,e} |
| | | q.d × 1 | |

^{a}Median % TGD increase compared to vehicle treated controls in time to 1 cc
^{b}p values vs. vehicle,
*< 0.05,
**< 0.01,
***< 0.001
^{c}p < 0.001 compared to docetaxel 33.3 mkd
^{d}p < 0.001 compared to HPM 100 mkd,
^{e}p < 0.001 compared to HPM, 75 mkd

### EXAMPLE 4

### Clinical Non-Small Cell Lung Cancer Study: HPM and Docetaxel

This study determined the MTD of ABT-751 when administered orally in combination with intravenous (IV) docetaxel in a NSCLC population. Following determination of the MTD, the study evaluated if the combination will prolong progression free survival (PFS) in subjects with NSCLC.

The primary objective of the Phase 1 portion of this study (conducted in the U.S. and Canada only) was to determine the MTD of ABT-751 when administered for 14 consecutive days in a 21-day cycle with standard docetaxel (75 mg/m²). The primary objective of the Phase 2 portion of the study was to assess if the addition of oral ABT-751 to standard docetaxel can prolong PFS compared to docetaxel alone in subjects with advanced or metastatic NSCLC. The secondary objectives of the Phase 2 portion of the study was to determine overall survival, time to disease progression (TTP), disease control rate, response rate, duration of response, quality of life, and characterization of the safety profile of ABT-751 when administered in combination with docetaxel.

All subjects received standard docetaxel (75 mg/m²) on Day 1 of each 21 -day cycle, via IV infusion over 1 hour. Oral study drug (ABT-751 or placebo [in the Phase 2 portion only]) was administered orally QD for 14 consecutive days followed by 7 days off drug. Dosing of ABT-751/placebo occurred with the start of the docetaxel infusion on Day 1 of each cycle.

As supplementation with dexamethasone began prior to docetaxel administration, the Screening Visit occurred between 2-14 days prior to Study Day 1 (i.e., the first day of docetaxel and study drug administration).

Study visits and chemistry laboratory tests were conducted weekly through Cycles 1 and 2, and then prior to the first dose for all additional cycles administered. Hematology tests were performed at Screening and weekly during docetaxel administration, as recommended in the docetaxel label.³ Urinalysis tests were performed prior to the first dose of each cycle.

A subject demonstrating a partial response (PR), complete response (CR), or stable disease (SD) continued to receive docetaxel and ABT-751 or placebo for as long as the subject was deemed to be clinically benefiting from treatment and any side effects were manageable. Oral study drug was continued as a single agent in these subjects following the completion of docetaxel therapy (as determined by the investigator) until disease progression or toxicities prohibited further continuation. In addition, subjects who completed docetaxel therapy but choose not to continue oral study drug or subjects who discontinued oral study drug due to toxicity remained on study for scheduled tumor assessments until progressive disease was determined or another antitumor therapy was initiated.

When an investigator determined that a subject should discontinue the study, a Final Visit was conducted. If the subject had not exhibited progressive disease and more than two weeks elapsed since the last tumor assessment, every effort was made to re-scan the subject prior to discontinuation.

All subjects had one Follow-up Visit approximately 30 days after the last dose of study drug. If the subject discontinued the study due to toxicities attributable to study drug, additional Follow-up visits were conducted at least every 30 days until the toxicity diminishes to an acceptable level or until toxicity was felt to be stable or irreversible.

Radiographic tumor assessments were conducted after every 2 cycles of study drug and/or docetaxel administration. Response criteria was assessed using the Response Evaluation Criteria in Solid Tumors (RECIST)¹⁰ to determine response rate, disease control rate, TTP, and PFS, as defined in Section 5.3.1.4. In addition, the investigator evaluated the subject for evidence of disease progression at each visit.

Toxicities were graded at each study visit according to the National Cancer Institute (NCI) Common Terminology Criteria for Adverse Events (CTCAE) version 3.0.¹¹

Subjects had survival assessments every 2 months following study discontinuation of therapy for a period of up to two years.

A maximum of 20 subjects at up to 6 sites, in the U.S. and Canada, were enrolled in the Phase 1 dose-escalation portion of the study. The initial dose of ABT-751 in the Phase 1 portion of the study was 200 mg QD. The dose of ABT-751 was escalated by 50 mg increments and dose reduction occurred in 25 mg decrements. All subjects were included for analysis of safety data. Subjects in the Phase 1 portion of the study who were not evaluable were replaced. Evaluable subjects were defined as those subjects who:
Experienced a DLT, or
Completed at least one cycle (21 days) of dosing with >_{—}80% compliance on the 14-day dosing schedule.

Blood sampling for PK analysis of ABT-751, ABT-751 metabolites, and docetaxel plasma concentrations were conducted on Cycle 1, Day 1, at 0-hr (pre-dose) and following ABT-751 administration at 0.5, 1, 1.25, 1.5, 2, 3, 4, 6, 8, and 24 hours (prior to ABT-751 dosing on Study Day 2).

After the MTD had been determined, the Phase 2 portion of the study randomized 160 subjects at approximately 50 sites in a 1:1 ratio to either docetaxel+ABT-751 (80 subjects) or docetaxel+placebo (80 subjects). All participating sites were informed by Abbott of the MTD established in the Phase 1 portion of the study prior to enrollment of subjects in the Phase 2 portion of the study. Subjects received either oral ABT-751 or oral placebo on Days 1-14 of each 21-day cycle. All subjects received docetaxel on Day 1 of each cycle.

Subjects completed a quality of life questionnaire at Screening, on Day 1 of each cycle, at the Final Visit and approximately 30 days following completion of therapy.

For those subjects in the U.S. who consented, pharmacodynamic (PD) samples for analysis of circulating tumor cells (CTCs) were collected at Screening, after Cycle 1, and at the Final Visit. Pharmacodynamic samples for proteomic analysis were collected for all consenting subjects at Screening, after Cycles 1 and 2, and at the Final Visit.

The date when the 110th confirmed event of progression occured or 3 months after last patient enrolled, whichever came later, defined the completion of the study. During the data collection period, active subjects continued to receive blinded study drug and docetaxel, if applicable.

When the data collection period was completed the study blind was broken. Any active subjects had a Final Visit and subjects randomized to ABT-751 plus docetaxel continued to receive open-label ABT-751 and docetaxel, if applicable, through an appropriate mechanism (e.g., single-patient IND or an extension protocol) until disease progression. Overall survival was collected on all subjects for up to 2 years after they discontinued the study, and this data was entered into the clinical database following the completion of the survival collection period.

### EXAMPLE 5

### Clinical Study of HPM and Docetaxel in Patients with Metastatic Hormone Refractory Prostate Cancer

ABT-751 was self administered orally for 14 days starting on day 1 followed by a 7-day +/−1 day) rest period. Docetaxel was administered by a 1-hour intravenous infusion on day 1. Each 21 day (+/−1 day) period will be considered 1 cycle. Patients were treated according to the Dose Escalation table below starting at dose level 1. There was no intra-patient dose escalation. ABT-751 was taken immediately after the end of docetaxel infusion.

If a patient missed a dose of ABT-751 and less than 12 hours had passed since the scheduled dosing time, then the dose was taken immediately. If more than 12 hours passed since the dosing time, the patient skipped that day's dose, and took the next dose at the regularly scheduled time the next day. If a patient vomited within 15 minutes of taking a dose of ABT-751, then the patient took another dose to make up for it. The dose was only repeated once. If more than 15 minutes has passed from the time the patient took a dose to the time they vomited, then the dose was not repeated.

Dose-limiting toxicity (DLT) is defined as drug related NCI CTC v3.0 grade 3 or 4 nonhematologic toxicity (except nausea or vomiting), or hematologic toxicity defined as any grade 4 thrombocytopenia or grade 3 thrombocytopenia with bleeding, or neutropenia defined as Grade 4 toxicity lasting for >5 days duration, or febrile neutropenia. It is also considered a DLT if a patient receives less than 50% of the intended dose of ABT-751 because of treatment related toxicity. Dose-limiting toxicity is defined on the first cycle

TABLE 9
Dose Escalation for Example 5 Clinical Study

| | ABT-751 (mg) | | |
|---|---|---|---|
| | Daily × 14 days | Docetaxel (mg/m²) | |
| Dose level | every 21 days | Day 1 every 21 days | Number of Patients |
| −2 | 75 | 50 | 3-6 |
| −1 | 100 | 50 | 3-6 |
| 1 (Start) | 100 | 60 | 3-6 |
| 2 | 150 | 60 | 3-6 |
| 3 | 200 | 60 | 3-6 |
| 4 | 200 | 75 | 3-6 |

only for doseescalation to the next level but cumulative toxicity will be noted.

If 1 out of 3 patients at any level experienced DLT, additional patients were entered at that level to a maximum of 6 patients. If 1 out of the 6 patients experienced DLT, then dose escalation was continued. If 2 out of the 6 patients experienced a DLT, that dose was then declared the maximum tolerated dose (MTD), and dose escalation was stop. If >2/3 or >2/6 patients exhibited a DLT, then the MTD was exceeded, dose escalation was stopped and the next lower dose was declared the MTD. A recommended dose was determined from the toxicity and pharmacokinetic data. If the MTD was dose level 1, then dose de-escalation occurred and patients were accrued to dose level −1.

Once a recommended dose was defined, the dose level was expanded so that a total of up to 20 chemo-naive patients with HRPC were enrolled to that dose level to further define toxicity and preliminary anti-tumor activity in that patient population.

After removal from protocol treatment, all patients were followed at one month then monthly until resolution of treatment related toxicities except alopecia and peripheral neuropathy grade 2 or less. Patients with documented response or stable disease were followed every 3 months with the relevant diagnostic imaging and laboratory tests until relapse/progression for duration of response/stable disease. Patients with documented PSA response or stable disease had monthly PSA performed until confirmed progression.

### EXAMPLE 6

### Preclinical Non-small Cell Lung Cancer Study: HPM and Cisplatin

In a non-small cell lung cancer study, tumor cells were inoculated s.c. into male nude mice on day 0. On day 10, mice bearing established tumors were size matched at about 233 mm³ and placed into the following groups:
1. HPM-100(p.o., q.d., 5 days on, 5 days off ×2)
2. PM-75 mg/kg/day (p.o., q.d., 5 days on, 5 days off ×2)
3. PM-0 mg/kg/day (p.o., q.d., 5 days on, 5 days off ×2)
4. Cisplatin 10 mg/kg/day (ip, qd ×1)
5. Cisplatin 0 mg/kg/day (ip, qd ×1)
6. Combination Therapy-HPM 100/Cisplatin 10 mg/kg/day (p.o., q.d., 5 days on, 5 days off ×2/i.p., q.d. ×1)
7. Combination Therapy-HPM 75/Cisplatin 10 mg/kg/day (p.o., q.d., 5 days on, 5 days off ×2/i.p., q.d. ×1)
8. Combination Therapy Vehicles

In the Calu-6 xenograft model, HPM administered as a single agent at 100 and 75 mg/kg/day on a (5 days on, 5 days off for 2 cycles) schedule demonstrated significant antitumor activity. In combination with cisplatin, HPM demonstrated additive responses with both doses of HPM tested. The results are shown in TABLE 10.

TABLE 10
In vivo efficacy of HPM alone and in combination with
cisplatin in the Calu-6 lung subcutaneous flank xenografts
grown in male nude mice.

| | Dose | Route | |
|---|---|---|---|
| Compound | (mg/kg/day) | Schedule | % TGD^{a} |
| HPM | 100 | p.o., q.d., (5 days on, 5 | 65***^{b} |
| | | days off) × 2 | |
| | 75 | | 58*** |
| Cisplatin | 10 | i.p., q.d × 1 | 71*** |
| HPM/Cis | 100/10 | p.o., q.d., (5 days on, 5 | 188***^{c,d} |
| | 75/10 | days off) × 2/i.p., q.d × 1 | 158***^{c,e} |

^{a}Median % TGD increase compared to vehicle treated controls in time to 1 cc
^{b}p values vs. vehicle,
*<0.05,
**<0.01,
***<0.001
^{c}p < 0.001 compared to cisplatin 10 mkd,
^{d}p < 0.001 compared to HPM 100 mkd,
^{e}p < 0.001 compared to HPM, 75 mkd

### EXAMPLE 7

### Preclinical Colon Cancer Study: HPM and 5-FU

In a colon cancer model, tumor cells derived from serially passaged tumor fragments were inoculated s.c. in female nude mice on day 0. On day 10, mice bearing established tumors were size matched at about 236 mm³ and placed into the following groups:
1. HPM at 100 mg/kg/day (p.o., q.d., 5 days on, 5 days off ×2)
2. HPM at 75 mg/kg/day (p.o., q.d., 5 days on, 5 days off ×2)
3. HPM at 50 mg/kg/day (p.o., q.d., 5 days on, 5 days off ×2)
4. HPM at 0 mg/kg/day (p.o., q.d., 5 days on, 5 days off ×2)
5. 5-FU 40 mg/kg/day (i.p., qd ×1)
6. 5-FU 30 mg/kg/day (i.p., qd ×1)
7. 5-FU 20 mg/kg/day (i.p., qd ×1)
8. 5-FU 0 mg/kg/day (i.p., qd ×1)
9. Combination Therapy: HPM at 100 mg/kg/day (p.o., q.d., 5 days on, 5 days off ×2 and 5-FU at 30 mg/kg/day (i.p., q.d. ×1)
10. Combination Therapy: HPM at 75 mg/kg/day (p.o., q.d., 5 days on, 5 days off ×2 and 5-FU at 30 mg/kg/day (i.p., q.d. ×1)
11. Combination Vehicles

In the HT-29 colon xenograft model, HPM administered as a single agent at 100 and 75 mg/kg/day on a 5 days on, 5 days off schedule for 2 cycles, demonstrated significant antitumor activity. In combination with 5-FU at 30 mg/kg/day, HPM at the either 75 or 100 mg/kg/day demonstrated additive responses.

The results are shown in Table 11.

TABLE 11
In vivo efficacy of ABT-751 alone and in
combination with 5-FU in the HT-29 colon subcutaneous flank
xenografts grown in male nude mice. Tumors were size matched
at ˜236 mm³ and therapy was initiated.

| | Dose | | Tumor | |
|---|---|---|---|---|
| | (mg/kg/ | Route | Volume^{a} | |
| Compound | day) | Schedule | Day 38 | % TGD |
| HPM | 100 | p.o., q.d., (5 days on, | 619 ± 40 | 75*** |
| | | 5 days off) × 2 | | |
| HPM | 75 | | 942 ± 46 | 50*** |
| 5-FU | 30 | i.p., q.d × 5 | 525 ± 35 | 75*** |
| HPM/5-FU | 100/30 | p.o., q.d., (5 days on, | 127 ± 14 | 150*** |
| | 75/30 | 5 days off) × 2/i.p., | 433 ± 38 | 100*** |
| | | q.d × 1 | | |

^{a}Mean ± SEM
% TGD increase compared to vehical treated controls in time to 1 cc
p values vs. vehicle,
*<0.05,
**<0.01,
***<0.001

What is claimed is:

1. A method of treating cancer in a human comprising administering a therapeutically effective amount of N-(2-((4-hydroxyphenyl)-amino)pyrid-3-yl)-4-methoxybenzenesulfonamide and an antimitotic agent.

2. A method of treating cancer according to claim 1 wherein the in the antimitotic agent is a taxane.

3. A method of treating cancer according to claim 2 wherein the in the antimitotic agent is docetaxel.

4. A composition comprising a therapeutically effective amount of an antimitotic agent and N-(2-((4-hydroxyphenyl)amino)pyridin-3-yl)-4-methoxybenzenesulfonamide.

5. A composition according to claim 4 wherein the antimitotic agent is a taxane.

6. A compostition according to claim 5 wherein the taxane is docetaxel.

7. A method of treating non-small cell lung cancer in a human comprising administering a therapeutically effective amount of N-(2-((4-hydroxyphenyl)-amino)pyrid-3-yl)-4-methoxybenzenesulfonamide and docetaxel.

8. A method of treating metastatic hormone refractory prostate cancer in a human comprising administering a therapeutically effective amount of N-(2-((4-hydroxyphenyl)-amino)pyrid-3-yl)-4-methoxybenzenesulfonamide and docetaxel.

9. A method of treating cancer in a human comprising administering a taxane drug followed by a therapeutically effective amount of N-(2-((4-hydroxyphenyl)-amino)pyrid-3-yl)-4-methoxybenzenesulfonamide.

10. A method of treating cancer according to claim 9 wherein the taxane drug is docetaxel.
